(19) United States
(12) Patent Application Publication
(10) Pub. No.: US 2009/0017047 A1
(43) Pub. Date: Jan. 15, 2009

(54) Preparation for the Prevention and Treatment of Stress Conditions as Well as Functional and Organic Disorders of the Nervous System and Metabolic Disorders

(76) Inventor: Egon Tech, Rostock (DE)

Correspondence Address: Duane A Stewart III;BUCHANAN INGERSOLL & ROONEY, One Oxford Centre 20th Floor, 301 Grant Street, Pittsburgh, PA, 15219, US

(21) Appl. No.: 11/629,246

(22) PCT Filed: Jun. 10, 2005
(86) PCT No.: PCT/DE2005/001073

§ 371 Date: Aug. 20, 2008

(30) Foreign Application Priority Data
Jun. 11, 2004 (DE) 20 2004 009 180.1
Jun. 18, 2004 (DE) 20 2004 009 689.7

(51) Int. Cl. A61K 39/00; A61K 33/00; C01B 33/12; A61K 36/886

(52) U.S. Cl. 424/184.1; 424/722; 424/744; 423/335

(57) ABSTRACT

Disclosed is a preparation for preventing and treating stress conditions as well as functional and organic disorders of the nervous system and metabolic disorders, to be used by people suffering from actinic dermatitis, against sunburn, as a regenerative agent for damaged cells/cell systems, and for the well-being of humans and animals. Also disclosed are methods for isolating cell components from Aloe barbadensis miller, producing energized/magnetized microparticles and nanoparticles, and using cell components. Previously known substances that arm used for the indications mentioned above are expensive to produce or have insufficient effective properties. Preparations containing glycine, especially in a gel formulation, are easy to produce while being surprisingly advantageous for the most various applications. As a result of the excellent properties of glycine, the inventive preparations are easy to implement for external and/or internal uses, providing effective prophylaxis and possibilities for beating the different affected body zones or improving the perceived state.

The invention relates to a preparation containing glycine for the prevention and treatment of stress conditions as well as functional and organic disorders of the nervous system and metabolic disorders and also for external application in sun allergies and against sunburn, in particular in inflammatory reactions of the skin due to UV-A, UV-B and UV-C rays of the sun or artificial sources of light and stressors which accelerate the aging process of the cells/cell systems and also for external and internal application for stress minimization in persons who are particularly subjected to psycho-emotional stress (profession, work, school, studies, sport, top-class sport, pre-menstrual/menstrual/post-menstrual stress, PMSD), to increase the libido, to increase the erection (in cases of stress-induced deficits), for more balance in old age, to weaken hyperactivity in adults and children, to increase defensive powers against pollutant and environmental toxins, electro-smog etc. and also for the prevention and accompaniment of the therapy of addiction diseases.

The recipe can also be used to minimize stress in animals (domestic animals and pets, exotic animals).

Fields of application of the invention are medicine and pharmacy.

Glycine has the molecular formula H₂N-CH₂COOH and is the simplest (non-essential) amino acid. Glycine is formed from serine (separation of activated formaldehyde by serine hydroxymethyl transferase), from L-threonine (acetaldehyde separation by threonine aldolase) or from glyoxylate (by transamination).

The function of glycine within the framework of synaptic signal transmission entails the inhibitory effect on the glycine receptors for regulation of intrinsic chloride channels in the spinal cord and cortex.

According to the latest knowledge, there are close connections between the effect principles of glycine and substance P and substance P fractions and their interaction. Ion channels are membrane proteins which traverse the cell membrane from the inside to the outside. The membrane itself is a very efficient diffusion barrier for ions. The hydrophobic inner area of the membrane prevents the access of ions and thus enables maintenance of ion gradients between cytoplasm and extra-cellular space. Ion channels possess a narrow pore, through which the ions can pass out of the cell or into the cell. The center of the signal transmission on chemical synapses is formed by the so-called ligand-controlled ion channels. The ligands are termed as neuro-transmitters, which bind onto assigned receptors on the surface of a target cell as chemical messengers.

Integral parts of the channels are mainly a number of protein segments. These trans-membranal segments comprise a high number of hydrophobic and a low number of hydrophilic amino acid residues, with polar and charged amino acids predominating outside the membrane.

The pore is opened for the passage of a certain ion at the moment at which a ligand docks onto a specialized protein domain (ligand binding point), which leads to a change in conformity of the pore.

In channels opened by neuro-transmitters, the ligand binding points (receptors) are on the outside of the cell.

Here, the receptors for the neuro-transmitters acetylcholine, glutamate, glycine, y-amino butter acid and serotonine are positioned. All these receptors have a homologous structure and are put together to form a super-family. The best examined representative of this group is the nicotinic acetylcholine receptor, which is mainly used as an explanation model.

The aforementioned neuro-transmitter have an ion selectivity, i.e. when the transmitter docks onto the receptor, the ion channel only becomes permeable for certain ions. For example, acetylcholine opens the passage of sodium, potassium and calcium ions on the motoric end plate of the muscles or the autonomous ganglia and leads to an activation of the target cells (exciting effect, depolarization of the membrane).

The neuro-transmitter glycine regulates the passage of chloride ions and hydrogen carbonate ions above all in the nerve cells of the spinal cord and the brain stem and has an inhibitory effect on the influenced nerve cell (inhibitory effect, hyperpolarization). Channels controlled by cytoplasmatic ligands have binding points for cellular messengers on the inside of the membrane (guanine adenosine phosphate-controlled).

The strychnine-sensitive glycine receptor (specific antagonist strychnine) as a ligand-controlled chloride channel of the post-synaptic membrane is an important transmitter of synaptic inhibition in the central nervous system. In this context, glycine is above all involved in the neuronal regulation of the muscle tonus through the centers in the spinal cord and brain stem.

Likewise, the glycinergic inhibition also contributed to the development of the breathing rhythm in the respiratory centers of the brain stem.

As a result of the loss of the glycinergic inhibition, excitatory impulses can develop without obstacles, e.g. in a strychnine intoxication. The neuro-muscular disinhibition results in painful tetania, which are further reinforced by sensoric stimuli. This means that, for example, a disorder of the glycinergic interneurons of the spinal cord (Renshaw cells), which form a control circuit together with the motoneurons of the skeletal muscles, leads to a deregulation of the skeletal muscles with the development of painful cramps.

These disorders within the function of the chloride channels, which can also be hereditarily induced, is also termed ion channel diseases (channelopathies). They are manifested in hypertonic disorders of movement. A known example is represented by the clinical picture of the Myotonia congenita.

Other agonists in the glycine receptors are also the amino acids β-alanine and taurine.

Stress is currently a term used frequently in everyday life by humanity, from small children up to old age, and in all languages. At the same time, there are extraordinarily divergent opinions on this term with experts and laymen, but also on the prevention, therapy, diagnostics and experiencing of stress.

Below, a holistically aligned opinion based on regulation theory is portrayed.

Observing man in his bio-psycho-physiological unit with reference to regulation theory knowledge, according to which strains and effects cause changes in regulation in the sense of a strain (bar theory) and each being is subject to a waking/sleeping cycle, one terms emotional stress as a temporary or permanent change of the individual psycho-physiological homeostasis.

Emotional stress represents a complex holistic function. The stress-triggering stimulus is termed the stressor, which can come from both the exogenous as well as the endogenous milieu. Emotional stress can express itself in two forms:

### EUSTRESS=PHYSIOLOGICAL FUNCTION

### DISSTRESS=PATHOLOGICAL VARIETY OF STRESS

Eustress supports health, motivation to perform and adaptability of the individual. The relationship between eustress and performance is not linear, but is subject to a bell-shaped course of the curve (Yerkes-Dodson's law).

The latest state of knowledge on stress presupposes that there is a provision of life energy via vegetative, metabolic and hormonal systems (normally in excess) during the emotional eustress, in order to carry through the adaptation to amended environmental conditions or to do justice to requirements and strains.

After the end of emotional stress, all the functions adjust to the normal flowing speed again (homeostasis). If the return does not take place by relaxation, for example, emotional disstress can occur, through which health damage can be caused in the event of lastingness. The origination of disstress, the pathological form of emotional stress, depends on a number of factors, with disorders of the emotions (conflicts, suppressed emotions, fear and others) and time regulation (effects against the biological clock) as well as mental under-load being able to exercise a strong pathogenic effect according to the latest knowledge.

Disstress is the promoter for 80 percent of all illnesses, including cancer, allergies, AIDS, cardiovascular diseases and psychic disorders. In many countries, disstress is disorder factor no. 1 for the quality of life, ability to perform and health (e.g. USA, Japan, Germany, Switzerland). Nowadays, most people and even children are disstressed, psychically and physically cramped, tense or hypertensive and also aggressive. As a result, they cannot go to sleep, suffer a reduction of performance and commit misperformances. Many people have inner unrest, a tense psyche and body, without them being conscious of this. These tensions are reflected in head, neck and back pains without organic damage existing. Mental talking to oneself, pondering, negative thinking also disstress and involuntarily prevent relaxation.

In Germany and in Europe, one in four people suffer from disstress-induced disorders of going to sleep. One in three feels tired during the day. Chronic disstress however has a further property which is not taken into account much, but is decisive for the self-estimation of being stressed. As a result of chronic disstress, critical ability, perception of symptoms, self-estimation with regard to load-bearing capacity, ability to make decisions, assessment of situations affecting one's own person and inter-personal life are lost as a result of the release of messengers, e.g. endorphins. Examinations show that of more than 100 people

only 19 percent can estimate their being stressed in reality,
58 percent underestimate their being stressed and
23 percent underestimate their being stressed.

A health threat exists above all in those who underestimate their being stressed. They need help by specific technical warning systems at all costs, i.e. a distress warning device and directly accessible bio-feedback systems, which are objectively able to control relaxation, and also systems which display or signalize that a break is necessary every 90-100 minutes.

Further examinations show that only one in six is in a position to estimate his ability to relax in reality. With the other 5, there is an overestimation to a great extent, i.e. the belief that one is relaxing, which is not the case in reality. However, this ability can be learned with bio-feedback systems.

In addition, disstress has a decisive influence on the quality of life. The scale for the assessment is the new version of the definition of health (Ottawa Charter) of the World Health Organization (WHO), which demands a corresponding measure of psychic, social, physical and economic well-being and ability to support oneself into old age. An objective monitoring system on the basis of measured vital parameters does not exist up to now.

The task of the present invention entailed developing substances or mixtures of substances which can be used for
a) prevention and therapy of stress conditions,
b) prevention and therapy of functional and organic disorders of the nervous system,
c) prevention and therapy of metabolic disorders,
d) binding of toxic substances,
e) physiological activation of inhibition processes in the CNS,
f) prevention and therapy of psychic disorders,
g) prevention and therapy of the syndrome of the reduction of ability to work and psychic over-stress.
h) developing substances or mixtures of substances which
   possess an inflammation-inhibiting effect on the skin,
   have a positive influence on the irritations of the nerve cells to be found in the layers of the skin and connected with it and thus contribute to an improvement of well-being, and
   finally support the restoration of the standard condition and also
   result in a reliable, long-lasting prevention against the aforementioned forms of damage.

In the course of the invention, it was seen that glycine also shows an effect as a co-transmitter on the excitatory NMDA receptors (N-methyl-D-aspartate receptors) by supporting the opening of this ion channel induced by glutamate via a specific binding domain.

The NMDA receptors also play a specific role in excitatory signal transmission and the development of the nervous system and also in diseases of the CNS. Apoplexy and other central nervous disorders, caused by neuro-toxic cell destructions, are the result of the excessive activation of NMDA receptors. Excessive excitation conditions and other psychic diseases are also to be assigned here.

According to the invention, it was seen that a positive influence of the NMDA receptors can be brought about by the external provision of glycine.

It proved to be a surprise that there is a very quick uptake of glycine into the central nervous system by the application of glycine in a very low application dosage, which leads to an effective, positive influence of the nervous metabolism.

This results in a very quick inhibition of central nervous disorders, the relief of stress situations, changes of psychosomal adaptation and improvement of the personal ability to perform.

### Pharmacological Properties:

The preparation according to the invention is a metabolic regulator which normalizes processes of excitation and inhibition in the CNS, possesses an anti-stress effect and increases the intellectual ability to work.

### Application—Indication:

The preparation can be administered on healthy children aged one year, juveniles and adults in order to increase the intellectual ability to work, in stress situations, in psycho-emotional tension (examinations, conflicts etc.). As an anti-stress agent and a nootropic agent, it can be applied on children older than 1 year, juveniles (also those with changes of their behavior), adults with various functional and organic diseases of the nervous system (neuroses, neurotic conditions and vegetative dystonia, in functional and organic consequences of cerebral traumata, in various forms of encephalopathy, amongst them alcoholic pathogenesis), which are accompanied by increased irritability, emotional instability, limitation of the intellectual ability to work and sleep disorders.

### Application Method and Dosage:

Glycine is preferably applied orally (gel), intra-nasally (as a brine/gel formulation) or vaginally (gel), particularly preferably sub-lingually (placed under the tongue) in a tablet form/gel form of 0.1 g at a time. In factually healthy children, juveniles and adults, the preparation is to be administered in dosages of 2-3×1 tablets/day for the term of 14-30 days in the event of weakness of memory, limitation of attention and concentration, in limitation of the intellectual ability to work as well as inhibition of development of the intellectual ability to work and in changes of forms of behavior in children and juveniles. In this way, the daily dosage amounts to 0.3 g.

In the event of psycho-emotional tensions, the preparation is used in dosages of 2-3×1 tablet per day for a duration of 14-30 days.

In functional and organic consequences of traumata of the nervous system accompanied by increased irritability, emotional instability and sleep disorders, dosage is to be as follows:

Children up to the age of 3: ½ tablet (0.05 g) 2-3× a day for the duration of 7-14 days, ½ tablet further being administered 1× per day for a duration of 7-10 days.

Daily dose: 0.1-0.15 g. Therapy cycle dosage: 2.0-2.6 g glycine.

Children older than 3 and adults are to be given 2-3×1 tablet/day for the duration of 7-14 days. If required, the therapy cycle can be repeated.

For sleep disorders, glycine is to be administered 20 minutes before going to bed or immediately before in a dosage of ½ tablet (as a function of the age).

In narcology, glycine is dosed as follows as an agent increasing the intellectual ability to work and as an agent against psycho-emotional tension during remission of encephalopathological phenomena and in the event of organic changes of the central and peripheral nervous system:

2-3× a day for the duration of 14-30 days. If required, such a treatment cycle can be repeated 4-6× a year.

The preparation does not reinforce the effects of sleeping tablets and alcoholic beverages.

No negative effects of glycine with other applications of drugs have been established. No undesired side-effects have been established.

Main indications for glycine are:
prevention and therapy of stress conditions,
functional and organic disorders of the nervous system. Restoration of these functions is achieved with the presence of amino-acetic acid as an active agent. (glycine, glycocoll)
Inhibition mediator in interaction with glycinergic receptors of the spinal cord and the brain, thus contributing to normalization of the balance between excitatory and inhibitory neuro-transmitter systems. In addition, glycine has the ability to bind various endogenous toxic substances (neutralization: phenols, aldehydes, barbiturates and others), thus making it possible to apply glycine as a therapeutic and prophylactic as well in areas with unfavorable ecology (environmental strains).

### Benefits of Glycine:

In its effect mechanism and pharmacological effect, glycine has no analogues (combines anti-stress, stress protection and nootropic effects inside itself).
The effect is achieved by physiological activation of inhibition processes in the CNS.
Glycine has a quick pharmacological effect. (The preparation acts after as little as 5-10 minutes and requires no substitute therapy.)
Glycine is administered in doses 10 times smaller than other nootropic preparations.
Contraindications and undesired side-effects have not been proven.
Over-dosage of the preparation is not possible.
Unlike tranquilizers, no addiction or withdrawal symptoms have been observed in long-term consumption of glycine.
Glycine can be taken at any age (“family preparation”).

### Indications for Glycine:

### Diseases of the Nervous System e.g.:

disorders of cerebral circulation (disorders of circulation of the blood in the brain),
residual phenomena of disorders of cerebral circulation (disorders of circulation of the blood in the brain),
ischemic insults,
residual phenomena of ischemic insults,
metabolism dystrophy diseases of the nervous system,
cranio-cerebral traumata,
residual phenomena of cranio-cerebral traumata,
concussion,
cerebrovascular insufficiency.

### Metabolic Disorders e.g.

with children in the period of intensive growth,
in phenomena of old-age.

### Psychic Disorders e.g.

abstinence phenomena,
alcoholism,
sleeplessness,
disorders in the perception of information,
coma,
disorders of the intellectual functions,
dementia,
weakness in concentration,
memory disorders,
aggressiveness,
behavioral disorders (excitations or inhibitions),
obsessive ideas, compulsive neuroses
emotional tensions,
pseudo-melancholy in old age,
irritability,
depressive conditions,
inner unrest,
emotional instability,
neurasthenia,
alcohol psychosis,
toxicomania,
encephalopathy,
juvenile and other behavioral disorders.

### Intoxications e.g.

benzol intoxications,
arsenic intoxications,
narcotic intoxications,
hypnotic and tranquilizer intoxications.

Further fields of use are prevention and therapy of the syndrome of reduction of the ability to work and psychic over-burdening as well as acute conditions during delivery (e.g. asphyxia of the fetus).

Other indications are also as an acute therapeutic in traumatic damage and strokes and also in risk patients from these groups as prevention and therapy, as well as use in Morbus Parkinson, Morbus Alzheimer and Morbus Crohn.

Alongside the aforementioned gel formulation, a large variety of other forms of application or of administration of the preparation according to the invention are possible, in particular sub-lingual, per os, as an inhalate, intra-nasal, via all the mucous membranes, skin, vaginal, rectal and/or in combination with an implant. In this context, administration in a tablet form is particularly suited.

In addition, similar gel-formers with the same effective mechanism as those described below can be used for the production of the preparation according to the invention, for example also Carbopol 974 P and PNC 400 (recipe: fundamental gel type C).

In other formulations, the zeolith described below is replaced by SiO₂ nano-particles, in particular Köstrosol. Both variants are possible.

Further, the dosage of the content of glycine can be increased to at least 10 times the figures stated.

Veterinary use: Use can be as in the human area, the dosage in small animals being the same as that quoted.

In large animals, the dosage should be at least 10 times that of the application stated, with application as a long-term adjuvant or as an implant under the skin or as a bolus also being possible.

In administration of pour-on preparations, a combination is possible, as is an aerosol treatment in large systems of industrial animal husbandry (fowl, cattle, pigs).

For administration via fodder, technological preparations are necessary, for example micro-encapsulation, gelling and coating (covering with high-molecular sugars, starch or micro-cellulose), in which the active agents are then released in the small intestine.

It was also surprisingly seen that a very fast uptake of glycine into the central nervous system comes about through the application of glycine via the mucous membranes, avoiding the first-pass effect, with only a very low application dosage, which leads to an effective positive influence on the nervous metabolism.

In this way, there is a very quick inhibition of central nervous disorders, reduction of stress situations, change of psychosomal tension and improvement of the personal ability to perform.

A gel preparation which passes on the active ingredient glycine in a portioned way for resorption when applied to the mucous membranes (oral, nasal and vaginal mucous membranes, rectum) has proven to be a particular effective form of application in this context.

An initial and deposit dose can be applied with a slight change of the carrier medium with regard to its adhesive capacity on the mucous membranes.

The foundation of the present invention is formed by gels with a high ion exchange capacity, high capacity to penetrate into the various layers of the skin and high adhesion on the skin and the mucous membranes. The core of the invention is a basic formulation which permanently repeats itself and can be mixed with other components.

Recipe for fundamental gel 1: TYPE C

| | |
|---|---|
| Sorbic acid | 0.10% |
| Glycine | 2.00% |
| Water, distilled | 73.80% |
| Carbopol 940 Pb. Eur | 0.60% |
| Sodium hydroxide, 10% sol. | 1.70% |
| Kollidone | 1.50% |
| Water distilled | 20.30% |
| Total | 100.00% |

### Manufacture:

Sorbic acid and glycine are dissolved hot in water. Carbopol is stirred in until a homogeneous mixture is achieved. After this, there is neutralization with NaOH (pH 5.0-5.5).

After this, the solution of kollidone and water is stirred in homogeneously.

For the hair/hair vitalization indication, the following ingredients are added to 100% type C:

| Fraction | Ingredient | Specification | | Quantity | |
|---|---|---|---|---|---|
| 1 | Aloe Vera Extract | 0-8 | kDa | 2.0 | ml |
| 2 | Extract manufactured acc. to | 0-10 | kDa | 2.0 | ml |
| | DE-OS | | | | |
| 3 | Extract manufactured acc. to | 10-30 | kDA | 0.5 | ml |
| | DE-PS | | | | |
| 4 | Extract manufactured acc. to | 60-90 | kDa | 1.0 | ml |
| | DE-PS | | | | |
| 5 | Alum | saturated | | 2.0 | ml |
| | | solution | | | |
| 6 | Aloe Vera in soy lecithin oil | | | 1.0 | ml |
| 7 | Zeolith (micronized/energetized) | 0.001 | mm Ø | 2.0 | g |

For the actinic dermatitis indication (inflammatory, allergic disease of the skin), the following ingredients are added to 100% type C:

| Fraction | Ingredient | Specification | | Quantity | |
|---|---|---|---|---|---|
| 1 | Aloe Vera Extract | 0-8 | kDa | 2.0 | ml |
| 2 | Extract manufactured acc. to | 0-10 | kDa | 2.0 | ml |
| | DE-OS | | | | |
| 3 | Extract manufactured acc. to | 10-30 | kDA | 0.5 | ml |
| | DE-PS | | | | |
| 4 | Extract manufactured acc. to | 60-90 | kDa | 1.0 | ml |
| | DE-PS | | | | |
| 5 | Alum | saturated | | 2.0 | ml |
| | | solution | | | |
| 7 | Zeolith (micronized/energetized) | 0.001 | mm Ø | 2.0 | g |

For the indication of sunburn before or after skin irritation by UV rays (and other out-of-the-ordinary stressors)

### Before Irritation (Prevention)

| Fraction | Ingredient | Specification | | Quantity | |
|---|---|---|---|---|---|
| 1 | Aloe Vera Extract | 0-8 | kDa | 2.0 | ml |
| 2 | Extract manufactured acc. to | 0-10 | kDa | 2.0 | ml |
| | DE-OS | | | | |
| 3 | Extract manufactured acc. to | 10-30 | kDA | 0.5 | ml |
| | DE-PS | | | | |
| 4 | Extract manufactured acc. to | 60-90 | kDa | 1.0 | ml |
| | DE-PS | | | | |
| 5 | Alum | saturated | | 2.0 | ml |
| | | solution | | | |
| 6 | Aloe Vera in soy lecithin oil | | | 4.0 | ml |
| 7 | Zeolith (micronized/energetized) | 0.001 | mm Ø | 2.0 | g |

### After Irritation (Therapy)

| Fraction | Ingredient | Specification | | Quantity | |
|---|---|---|---|---|---|
| 1 | Aloe Vera Extract | 0-8 | kDa | 5.0 | ml |
| 2 | Extract manufactured acc. to | 0-10 | kDa | 2.0 | ml |
| | DE-OS | | | | |
| 3 | Extract manufactured acc. to | 10-30 | kDA | 0.5 | ml |
| | DE-PS | | | | |
| 4 | Extract manufactured acc. to | 60-90 | kDa | 2.0 | ml |
| | DE-PS | | | | |
| 5 | Alum | saturated | | 2.0 | ml |
| | | solution | | | |
| 6 | Aloe Vera in soy lecithin oil | | | 10.0 | ml |
| 7 | Zeolith (micronized/energetized) | 0.001 | mm Ø | 2.0 | g |

For the indication of revitalization (regeneration of damaged cells/cell systems) of aging, mature skin for reduction of wrinkles and tightening:

| Fraction | Ingredient | Specification | | Quantity | |
|---|---|---|---|---|---|
| 1 | Aloe Vera Extract | 0-8 | kDa | 4.0 | ml |
| 2 | Extract manufactured acc. to | 0-10 | kDa | 1.0 | ml |
| | DE-OS | | | | |
| 3 | Extract manufactured acc. to | 10-30 | kDA | 0.5 | ml |
| | DE-PS | | | | |
| 4 | Extract manufactured acc. to | 60-90 | kDa | 0.5 | ml |
| | DE-PS | | | | |
| 5 | Alum | saturated | | 0.5 | ml |
| | | solution | | | |
| 6 | Aloe Vera in soy lecithin oil | Dry skin | | 1.0 | ml |
| | | type | | | |
| | | Greasy | | | |
| | | skin type | | | |
| 7 | Zeolith (micronized/energetized) | 0.001 | mm Ø | 2.0 | g |

Indication minimization of stress and improvement of well-being:

For this indication, intra-nasal application (also as an inhalate) is preferred. This form of application guarantees a quick start of effect. The effect commences within about 10 seconds and lasts for about 4 hours. The recipe can be post-dosed at any time (e.g. in the form of a nose spray). Over-dosage is not possible, which means that self-medication is absolutely free of risk and damage. The start of the effect makes itself noticeable with a shine in the eyes (approx. 10 sec. after administration, sometimes even more quickly).

Alongside intra-nasal application (also as an inhalate), the applications per os and/or sub-lingual can be considered. For example, the recipe is administered micro-encapsulated and can be used for a broad range of indications (Morbus Crohn, nervous stomach, stage fright etc.).

Use in the foodstuffs and consumables industry and also as a nutritional supplement are possible with the technological preparations described.

Recipe: Type C in its basic formulation, plus 2.0 g of zeolith (0.001 mm ø), plus 0.1 ml mint oil (refreshment and taste) Dosage: 0.18 ml per nostril Packaging: e.g. 3 ml in spray bottle or 20 ml in tube with nose adapter

A further possibility of application is applying the recipe with the help of a roller (e.g. with 7 ml contents) for relaxation and stress minimization, e.g. around the eyes, with a simultaneous refreshing effect which starts immediately (tired eyes effect after too little sleep or disco etc.) and an immediate start of a tightening effect around the eyes etc.

There is also the area for use as a lip gloss with stress-minimization properties and a simultaneous effect of furthering the blood circulation for larger and fuller lips.

A further possibility of use is as a cosmetic series for the female intimate area with contact to the mucous membranes and a quickly-starting effect (libido stimulation) or use as a surface coating in tampon manufacture (for better well-being in menstruation).

Further, use in the male genital area for local erection improvement is possible, although this should be preceded by oral and/or intra-nasal application.

### Use in the Veterinary Area:

The recipe is not only used on humans, but also for stress minimization in domestic animals, pets and exotic animals. Examples of this are cats, dogs, horses, camels, parrots, parakeets, hamsters and rabbits.

Use in animals can be with the same formulation and dosage, e.g. for dogs, cats, small animals and pet birds (e.g. 0.18 ml per cat or small dog, a larger dog being given 3-4 times the dose).

Administration via fodder, trough or in a spray procedure is also possible and can be used above all with fowl and large animals (horses, camels).

Use in animal nutrition (e.g. dogs and cats) is done for example as fodder, in which the active ingredients exist micro-encapsulated (gelled) or coated.

Alongside various possible methods of production, the following method and the following use have proven to be surprisingly favorable for the manufacture of a recipe with the properties according to the invention and have additionally made further areas of use possible:
1. A method for obtaining cell membrane components from Aloe barbadensis miller with antibacterial, virucide, antimycotic, anti-inflammatory, regenerative and stress-minimizing effect on animal and mammal cells following out-of-the-ordinary stressing by UV-A/B/C and other artificial sources of light, and also for restoration and prevention of the aforementioned effects.
   The use of these vegetable ingredients is done as a replacement for the ingredients of animal cell lines and their manufacture in comparable modes of effect/indications for animal and mammal cells, in particular through the use of their ingredients with a molecular weight of 0-10 kDa and also larger than 10 kDa as well as larger than 100 kDa.
2. The use of cell components as originate in production/cellular breeding, in particular for the manufacture of virus vaccines, the manufacture of RIV particles or the manufacture of biomune, mainly as cell supernatants, and are rejected as waste.

Ad 1. The method according to the invention for the manufacture of cell membrane components of Aloe barbadensis miller and their use in animal and mammal cells damaged by out-of-the-ordinary stressors (UV-A/B/C and other artificial sources of light) is made clear by the following description.

The leaves of the aloe, to be harvested fresh, are subjected to a microwave radiation for a short time, which does not lead to destruction of the cell structures, but, only as an out-of-the-ordinary stressor, leads to the formation and release of immunologically relevant materials of the cell membrane. The latter are cleaned, filtered and applied for inner and also outer application in the required factions (0-10 kDa, 10-100 kDa and larger), depending on the indication, with the effective properties corresponding to those of mammal cells extracts obtained. The similar components can be achieved after 30 minutes of shock frosting of the freshly harvested leaves at −20° C. and extraction, filtration of the leaf juice/gel obtained in this way or by short-term (10 min.) radiation of the freshly pressed aloe juice/gel with UV light. The out-of-the-ordinary stressing results in this context in equally desired substances/mixtures of substances which have outstanding immunological and regulatory properties and are very similar, possibly identical to the effective mechanisms of those obtained from animal cell lines.

In application via the skin, outstanding effects were achieved by the aforementioned recipes, preventing damaging of the skin/mucous membrane and also regenerating the cells/tissue which had already been damaged in a short period, which is to be put down to the anti-inflammatory, regenerative effect of the cell components. In this context, a DNA repair effect is absolutely not to be ruled out and is the object of further examinations. Everything indicates that it is possible that the application is to be recommended for combating of degenerate cells (cancer).

With regard to application as a prevention against the aforementioned stressors, there were no inflammatory processes on the skin, e.g. after use as a sun gel. Although there was slight reddening, the inflammatory process did not take place. Intensive browning of the skin nevertheless resulted.

Ad 2: In cell breeding, e.g. for the manufacture of virus vaccines and in the production of RIV particles, and also for the production of biomune, the cell supernatants obtained are rejected as waste. This resulted in the possibility of processing these supernatants, which are known to occur following stressing of the cells, as immunologically valuable ones following filtration, extraction and purification and of using them as active ingredients containing the aforementioned ingredients, e.g. in dermatology or in immunology.

The immunological properties are identical to those of the aforementioned fractions. The known methods have never taken this “waste” into account up to now and always rejected it according to the state of knowledge of science up to that time. According to the doctrine according to the invention, the valuable substances are now obtained from them and used. This is done by filtration and purification of the aforementioned cell supernatants for use in the application. The indication is analogous to the known methods for the obtaining of components of 0-10 kDa and larger than 10 kDa with a similar kind of objective, indications and ingredients, in particular the component parts of all the fractions of similarly effective substances originating in ultra-centrifugation by shearing forces.

The invention also entails the following procedure for energetization of silicon nano-particles and/or zeoliths or other mineral substances/mixtures of substances put into application for use for optimization of the control processes of vegetable, animal and mammal cells/cell systems/organs, organ systems, organisms. The procedure is suitable for application in
vegetable cells via the leaf and also via the micro-climate in the root and capillary area,
animal cells for all external and internal applications,
mammal cells for all external and internal applications,
in particular as adjuvants in cosmetics and medicine, e.g. being applied as an allergy-free medium in products for the skin etc. fields of application, both minerals, zeoliths, bentonite etc., as preferred nano-particles for application on animal and mammal cells.

For production of energetized/magnetized micro and nano-particles for application on plants, bentonite/zeoliths, minerals were subjected to a certain dosage and frequency of microwaves according to the invention, which lead to a change of the crystalline structures and contribute as transmitters to the optimization of the plant growth, to the information for the vegetable cells/cell systems and counteract or cut out stressors to the extent that a physiological plant growth is possible despite an increased frequency of stressors. This is done both via the leaf as application and also for the root area in order to support the micro-climate in the root area. The result of the application established in a research institute (for wheat) was an additional yield of 2 dt/ha and, even more impressive, there was an out-of-the-ordinary increase of the protein contents, e.g. in wheat, of 2%.

This method is to be explained below with the example of the use of a magnetron with a frequency of 2,450 Megahertz (others are possible) and impulses depending on the indication, as well as the production of energetized/magnetized micro and nano-particles with SiO₂ for use in animals and humans.

Both zeoliths with a diameter less than 100 micrometers and also finished nano and micro-particles with SiO₂ of the “Köstrosol” product series (protected products) from the “Chemiewerk Köstritz” were used.

The particles used were subjected to various doses/impulses of microwaves, which lead to a change of the crystalline grid and to the absorption of information about the microwaves, which can vary depending on the destination and the indication.

Use of a magnetron with a frequency of 2450 Megahertz (others are possible) and impulses depending on the indication.

A specific Köstrosol with a particle diameter of 7 manometers was specifically used for the application via the skin/mucous membranes and is in a position to bind substances with a similar size loosely and to take them to the destinations on or in the organism as a transporter for the preferred substances and also in the aforementioned molecular weights of 0-10 kDa and larger than 10 kDa and also of the filtered supernatants and extracted components of the membranes, in particular Aloe barbadensis miller obtained with the help of the method described. This results in the transmission of the applied vibration on the one hand and the transport of immunologically desired particles. The indications entail all the inflammatory, allergic processes which are caused by out-of-the-ordinary stressors and are the object of the invention.

For the mode of effect of the preparation according to the invention, the following case descriptions are given as examples:

Woman aged 35: after years of complaints in the genital area, lower abdomen, right down to a forthcoming total resection of the uterus:

after application of the gel formulation vaginally and intra-nasally, improvement starting within one week, almost free of complaints and well-balanced (very happy) up to the present.

Child aged 11: hyperactive, uncontrollable, conspicuous/was supposed to be given Ritalin:

after application intra-nasally and per os (micro-encapsulated, gelled): appearance is well-balanced with occasional relapses, considerably better scholastic performances and alignment into family life.

Man aged 66: Parkinson, psychosis:

After application via the skin, considerably better sequence of the disease, considerably better communication and pleasure in contact.

Child aged 8: conspicuous conduct, lack of attention:

after application via the skin: balanced, inquisitive, pleasure in contact.

Woman aged 32 after consumption of synthetic drugs in addiction with all symptoms: after application intra-nasally and dermally or vaginally: after 4 weeks absolutely in the complete standard range, happy after 1 year.

Man aged 66 after EMF radiation:

Sleep disorder, skin irritations, cardiac arrhythmia, concentration weakness Permanent taking intra-nasally, per os, via the skin: no complaints since start of taking

Man aged 46: concentration weakness, unrest etc.:

After application intra-nasally, free of complaints after 14 days

Woman 46 climacteric with complaints in the uro-genital area, hot flushes, unbalanced, sleep disorder, no sex life:

After application vaginally, dermally: no complaints after 14 days, excellent libido.

Man 46 businessman in total stress, with all phenomena of burn out:

After 14 days of intranasal application: full ability to perform, good sleep, good sex life again.

Man, 44, flies a lot across continents, manager, jetlag

After application intra-nasally, no time problems, no sleep problems, increased intellectual performance, better libido.

Woman, 43, Morbus Crohn:

After vaginal application, considerable improvement, after additional intra-nasal, almost free of complaints with quite slight thrusts.

Young man, 23, student: concentration weakness:

After intra-nasal application: increase of the intellectual ability to perform

Mann, 46, merchant: Morbus minieri:

After permanent intra-nasal application, free of complaints up to the present with slight thrusts, would be unable to work otherwise.

Man, 38, self-employed businessman: erectile dysfunction by stress etc.:

After application locally and intra-nasally no problems with the libido, complete stress-bearing capacity

1. Preparation for prevention and therapy of stress conditions, functional and organic disorders of the nervous system and metabolic disorders and also for application in actinic dermatitis, sunburn, as a regenerative for damaged cells and cell system and for stress minimization and increasing well-being in humans and animals, wherein the preparation contains glycine, preferably a fundamental gel containing glycine.

2. Preparation according to claim 1, wherein the preparation entails the following components:
water, distilled,
glycine,
sodium hydroxide,
kollidone,
Carbopol 940 Ph. Eur,
Sorbic acid.

3. Preparation according to one of the aforementioned claims, wherein there exists the following composition:

| Component | Share of the total quantity in percent |
|---|---|
| Water, distilled | 89-98% |
| Glycine | 0.5-4.0% |
| Sodium hydroxide | 1.0-2.5% |
| Kollidone | 0.5-2.5% |
| Carbopo1 940 Ph. Eur | 0.1-1.1% |
| Sorbic acid | 0.01-1.0% |

4. Preparation according to one of the aforementioned claims, wherein there exists the following composition:

| Component | Share of the total quantity in percent |
|---|---|
| Water, distilled | 92-96.5% |
| Glycine | 1.0-3.0% |
| Sodium hydroxide | 1.2-2.2% |
| Kollidone | 1.0-2.0% |
| Carbopol 940 Ph. Eur | 0.3-0.9% |
| Sorbic acid | 0.05-0.5% |

5. Preparation according to one of the aforementioned claims, wherein there exists the following composition:

| Component | Share of the total quantity in percent |
|---|---|
| Water, distilled | 94.1% |
| Glycine | 2.0% |
| Sodium hydroxide | 1.7% |
| Kollidone | 1.5% |
| Carbopol 940 Ph. Eur | 0.6% |
| Sorbic acid | 0.1% |

6. Preparation according to claim 1 or 2, wherein there exists the following composition:

| | |
|---|---|
| Sorbic acid | 0.10% |
| Glycine | 2.00% |
| Water, distilled | 73.80% |
| Carbopol 940 Ph. Eur | 0.60% |
| Sodium hydroxide sol. 10% | 1.70% |
| Kollidone | 1.50% |
| Water, distilled | 20.30% |

7. Preparation according to one of the aforementioned claims, wherein the preparation is administered orally, intra-nasally, dermally or vaginally, rectally.

8. Preparation according to one of the aforementioned claims, wherein it exists in a tablet form or in a gel form.

9. Preparation according to one of the aforementioned claims, wherein each tablet contains 0.01 g to 1 g of glycine.

10. Preparation according to one of the aforementioned claims, wherein each tablet contains 0.05 g to 0.5 g of glycine.

11. Preparation according to one of the aforementioned claims, wherein each tablet contains 0.1 g of glycine.

12. Method for the production of the preparation according to one of the aforementioned claims, wherein glycine is inserted into pharmaceutical preparations with methods which are customary and known per se.

13. Use of the preparations according to one of the claims 1 to 11, wherein it is administered orally, intra-nasally, rectally or vaginally.

14. Use of the preparations according to one of the claims 1 to 11, wherein it is administered sub-lingually.

15. Use of the preparations according to one of the claims 1 to 11 for prevention and therapy of stress conditions.

16. Use of the preparations according to one of the claims 1 to 11 for prevention and therapy of functional and organic disorders of the nervous system.

17. Use of the preparations according to one of the claims 1 to 11 for normalization of the balance between excitatory and inhibitory neuro-transmitters in the spinal cord and the brain.

18. Use of the preparations according to one of the claims 1 to 11 for binding various toxic substances, in particular phenols, aldehydes, barbiturates and others.

19. Use of the preparations according to one of the claims 1 to 11 for physiological activation of inhibition processes in the CNS.

20. Use of the preparations according to one of the claims 1 to 11 for prevention and therapy of functional and organic disorders of the metabolism.

21. Use of the preparations according to one of the claims 1 to 11 for prevention and therapy of psychic disorders.

22. Use of the preparations according to one of the claims 1 to 11 for prevention and therapy of intoxications.

23. Use of the preparations according to one of the claims 1 to 11 for prevention and therapy of the syndrome of reduction of the ability to work and psychic over-strain.

24. Method for obtaining cell membrane components from Aloe barbadensis miller with antibacterial, virucide, antimycotic, anti-inflammatory, regenerative and stress-minimizing effect on animal and mammal cells following out-of-the-ordinary stressing by UV-A/B/C and other artificial sources of light and also for the restoration and prevention of the aforementioned effects, wherein the leaves of the aloe to be harvested freshly are subjected for a short time to a microwave radiation which does not lead to the destruction of the cell structures, but, as an out-of-the-ordinary stressor, only leads to the formation and release of immunologically relevant substances of the cell membrane and wherein the leaves are cleaned, filtered and applied in the required fractions (0-10 kDa, 10-100 kDa and larger) depending on the indication.

25. Use of cell components as they originate and are rejected as waste, preferably as cell supernatants, in production/cell breeding, in particular for the production of virus vaccines, the production of RIV particles and/or the production of biomune, for the production of a preparation according to one of the aforementioned claims.

26. Method for the production of energetized/magnetized micro and nano-particles, containing SiO₂, for use on plants, animals and man, in particular as a transmitter for optimization of plant growth, and to counteract stressors, wherein bentonite/zeoliths and/or minerals, in particular Köstrol, are subjected to a certain dosage and frequency of microwaves, if applicable by the use of a magnetron with a frequency of 2450 Megahertz (and other frequency ranges) and impulses, depending on the indication.
